(19) United States
(12) Patent Application Publication
(10) Pub. No.: US 2025/0131663 A1
(43) Pub. Date: Apr. 24, 2025

(54) Referencing of Anatomical Structure

(71) Applicant: Novarad Corporation, Provo, UT (US)

(72) Inventors: Wendell Arlen Gibby, Mapleton, UT (US); Steven Todd Cvetko, Draper, UT (US); Joel Lyon, American Fork, UT (US); Ryan West, Payson, UT (US)

(21) Appl. No.: 18/532,914

(22) Filed: Dec. 7, 2023

Related U.S. Application Data
(60) Provisional application No. 63/591,383, filed on Oct. 18, 2023.

(51) Int. Cl. G06T 19/00; A61B 34/20; G06V 10/24; G06V 10/44

(52) U.S. Cl. CPC G06T 19/006; A61B 34/20; G06V 10/245; G06V 10/44; G06V 2201/07

(57) ABSTRACT

Technology is described for referencing anatomical structure using an AR headset. The method can include registering a marker located on a distal anatomical structure. The marker may have a pose that includes a position and orientation in a 3D coordinate system of the AR headset. Another operation may be identifying a proximal anatomical structure to which the distal anatomical structure is connected. The marker may be registered at a second pose having a second position and second orientation. A joint pivot axis and angle may be determined with respect to the proximal anatomical structure by comparing the first pose and the second pose. The joint pivot axis may then be displayed, using the AR headset. The technology may also use a pointer device to automatically find an anatomic path and anatomic length.

### RELATED APPLICATION

This application claims priority to U.S. Provisional Application No. 63/591,383 filed Oct. 18, 2023, entitled “Referencing of Anatomical Structure” which is incorporated herein by reference.

### BACKGROUND

Mixed or augmented reality is an area of computing technology where views from the physical world and images from virtual computing worlds may be combined into a mixed reality world. In mixed reality, people, places, and objects from the physical world and virtual worlds become a blended visual and audio environment. A mixed reality experience may be provided through existing commercial or custom software along with the use of VR (virtual reality) or AR (augmented reality) headsets.

Augmented reality (AR) is an example of mixed reality where a live direct view (or an indirect view) of a physical, real-world environment is augmented or supplemented by computer-generated sensory input such as sound, video, graphics or other data. Augmentation is performed as a real-world location is viewed and in context with environmental elements. With the help of advanced AR technology (e.g. adding computer vision and object recognition) the information about the surrounding real world of the user becomes interactive and may be digitally modified.

### BRIEF DESCRIPTION OF THE DRAWINGS

FIG. 1 is a diagram illustrating an example of identifying and referencing a joint pivot axis in a knee.

FIG. 2 illustrates an example of a variable circle (or slice of a cylinder) that defines an isometric radius for ligamentous insertion into a patella from a hinge point of a femur.

FIG. 3 is a diagram illustrating an example of a first marker on a distal anatomical structure of the joint and a second marker on a proximal anatomical structure of the joint.

FIG. 4A illustrates an example of identifying a joint pivot axis or hinge point of a knee joint between the tibia and femur.

FIG. 4B illustrates another example of identifying a joint pivot axis of a knee joint between the tibia and femur.

FIG. 5 illustrates an example of tracking the tibia in a leg in extension and identifying a joint pivot axis of a knee joint between the tibia and femur.

FIG. 6 is an illustration of an example of an optical marker near the junction of the femur and tibia (distal femur).

FIG. 7 is a flowchart illustrating an example method for referencing anatomical structure using an AR headset.

FIG. 8 illustrates an example of referencing anatomical distance using a pointing device, a virtual line, an image data set and an augmented reality (AR) headset.

FIG. 9 illustrates an example of a sagittal view of a vertebra and pointing device of FIG. 7.

FIG. 10 illustrates an example of measuring anatomical distance for use in repairing a bone fracture.

FIG. 11 illustrates an example of a 3D scan or 3D image data set of a knee in an extended position with markers.

FIG. 12 is an example of a sagittal view of a knee in an extended or unbent pose.

FIG. 13 illustrates an example of bone segments that are identified near a joint of a person.

FIGS. 14 and 15 illustrate an example where the anatomical segments may be rotated around a joint pivot axis in the image data set.

### DETAILED DESCRIPTION

Reference will now be made to the examples illustrated in the drawings, and specific language will be used herein to describe the same. It will nevertheless be understood that no limitation of the scope of the technology is thereby intended. Alterations and further modifications of the features illustrated herein, and additional applications of the examples as illustrated herein, which would occur to one skilled in the relevant art and having possession of this disclosure, are to be considered within the scope of the description.

This technology may include the use of augmented reality (AR) systems to provide identification of anatomical features (e.g., joint pivot axes), guidance in medical procedures related to human joints (e.g., knee, elbow or hip surgery) and improve the ability to realign dislocated and/or broken portions of limbs or human anatomy. The guidance may enable a medical professional to determine the distance from a joint pivot axis or joint pivot point to another location on the bone so that a cut may be made in a bone in an appropriate location. Further, the length of an anatomical structure may be tracked and measured using an augmented reality (AR) headset and a medical image data set aligned with the visible human anatomy. While prior medical imaging can show the anatomy of a patient as aligned with the patient, this technology may go farther and inform a medical professional about the location of a joint pivot axis and may inform the medical professional about isometric locations for attaching ligaments around the hinge.

In one example, during a knee replacement surgery, the medical professional may need to know the length of the leg or portions of the leg. The medical professional may also want measurements to make a saw cut so that a leg being operated on is same length as the other leg. This technology further enables the medical professional find a joint pivot axis, a ball joint pivot point (e.g., hip) the top of the leg or other important anatomical locations that may be challenging to identify during a medical procedure. In another example, this technology may be used to measure the length of a femur by tracking back to the point of origin of the femur.

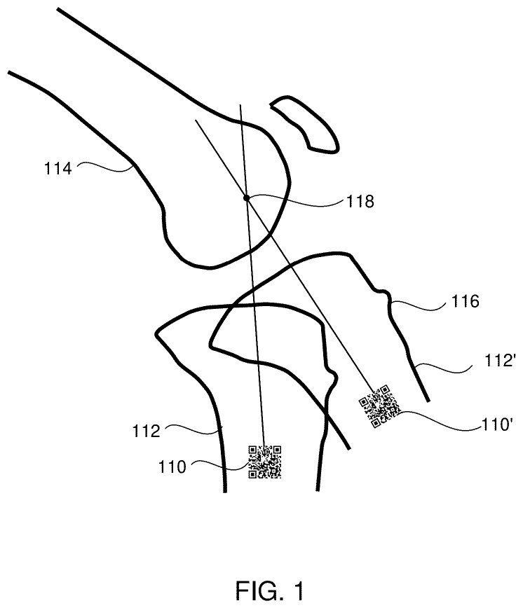

A system and method for referencing an anatomical structure using an AR headset may be provided. FIG. 1 illustrates an example of identifying and referencing a joint pivot axis in a knee. In order to identify a joint pivot axis point, a marker 110 may be registered on a distal anatomical structure 112. The marker 110 may have a pose that includes a position and orientation in a 3D coordinate system of the AR headset. The distal anatomical structure 112 may be the portion of a leg that is easily movable. A plurality of points can be registered on an optical code. This may allow a position and orientation of the marker to be determined for the pose and/or a center, corners or edges of the marker to be identified.

In addition, a proximal anatomical structure 114 may be identified to which the distal anatomical structure 112 is connected. The proximal anatomical portion 114 of the structure may be a portion of the leg that is attached to a patient's body. This proximal anatomical portion 114 may be considered fixed because the patient may be attached to an operating table or a gurney. The term fixed may also define that the proximal anatomical structure or portion does not move during a medical procedure due to some restraint or the weight of the proximal anatomical structure. The proximal anatomical portion of the patient may also be fixed when the patient is fixed in a position when lying or sitting down or in similar fixed positions. Alternatively, the distal anatomical structure of the person may be fixed and the proximal portion of the person may moved (e.g., using a lift, powered gurney, ctc.)

The marker 110 may also be detected at a second pose 116 that has a position and orientation with respect to the proximal anatomical structure 112′. The AR headset may receive a command from a user to detect the marker 110′ at the second position. In one example, the AR headset may be set to track the marker 110 continuously from the first pose until the marker stops at a second pose.

A joint pivot axis 118 may be determined by comparing the first pose and the second pose. The joint axis point 118 may then be displayed using the AR headset. In one example, the joint pivot axis 118 may be identified using an intersection of a first line that extends down a surface of the bone of the joint (or through an axis of symmetry of the bone) substantially parallel to the marker in a first pose and a second line that extends along a surface of the bone (or through an axis of symmetry of the bone) substantially parallel to the second pose of the marker.

In one example, a medical professional may put a marker 110 that is an optical tracker on an arm, leg or other anatomical structure of the patient while the patient is lying down. As the marker 110 is moved, a sensor in communication with the AR headset can find the new location of the marker 110′. Thus, the location of the joint pivot axis and a distance from the tracker 110 to the joint pivot axis can be computed.

The term pose may refer a position and orientation of a single marker in a 3D coordinate system or with reference to another marker. Because the marker may have a 2D area and distinguishing patterns on the 2D area, then multiple points on the marker can be registered and used to determine the position and orientation of the marker. When a single marker's poses are compared for two different positions (e.g., two different times), the second pose may be analyzed in relation to the first pose. The comparison includes computing a positional and rotational change from pose one to pose two (e.g., time one to time two). For example, the movement of a marker from point one to point two can be measured and then the marker rotation in degrees about the marker's center may be measured and displayed. The rotational component for comparing those two poses will be around a marker's axis. The rotation of the marker may be around an axis through the marker's center, but the rotation may also be a rotation around any other parallel axis (which rotation would also cause a positional change).

A pivot axis that accounts for the positional change of the marker in the plane perpendicular to the axis of rotation may also be determined. That pivot axis corresponds to the joint pivot axis as long as the marker is attached to a rigid part that is pivoting (e.g., a bone). There may also be some movement not accounted for that is parallel to the pivot axis, and this may be displayed as a displacement of the bone occurring with the rotation. Computing the joint pivot axis with a single marker assumes that at least one portion (e.g., the proximal portion) of the joint is fixed in 3D space. In another configuration without that assumption, a second marker may be used on the other side of the joint, and computations may be in relation to a moving-coordinate system containing both proximal and distal anatomical structures of the joint (instead of a fixed-world system).

The distal anatomical structure and the proximal anatomical structure may include bones forming the joint. In one example, the proximal anatomical structure may be a femur and the distal anatomical structure may be a patella or tibia. Other pairs of bones may also be identified as the distal anatomical structure and the proximal anatomical structure. For example, the humerus may be a proximal anatomical structure and the radius or ulna may be used as distal anatomical structure.

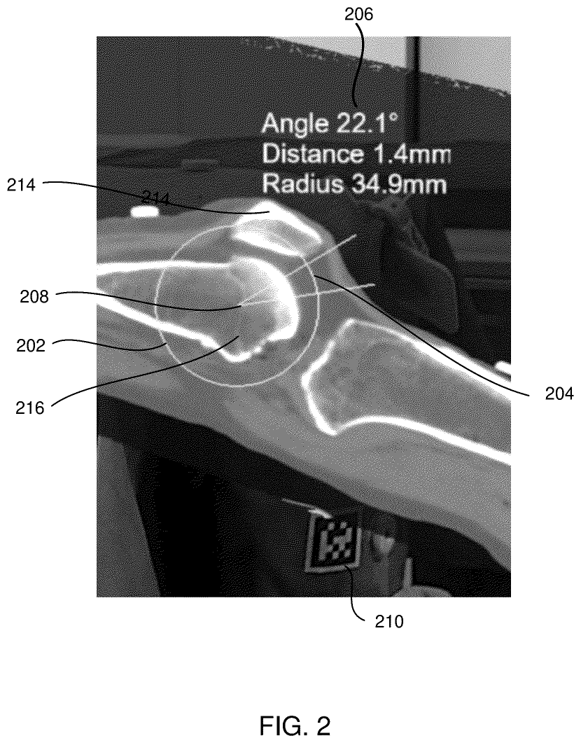

FIG. 2 illustrates an example of a variable circle or cylinder that defines an isometric radius for ligamentous insertion into a patella 214 from a joint pivot axis 208 (or hinge point) of a femur 216. A plurality of points 202 may be displayed in FIG. 2 that surround the joint pivot axis 208 and are isometric to the joint pivot axis 208. In addition, a computed angle of rotation 204 may be displayed. The plurality of points 202 may form a circle as illustrated that is a part of or a slice of a cylinder that extends perpendicular to plane illustrated by FIG. 2. The circle, variable circle or cylinder is a virtual circle or virtual cylinder that is provided as an overlay in an AR headset for viewing by a medical professional or user. The plurality of points may form a virtual display object that is at least one of: an arc, a circle, a cylinder, a curved surface, or an irregular shape. In the addition, the arrangement of the points does not need to be continuous, as illustrated, but may be one or more disjoint points. The plurality of isometric points 202 surrounding the joint pivot axis 208 can be displayed based in part on a defined ligament length in order to identify locations on a bone (e.g., patella, femur, tibia, humerus, radius, etc.) where a ligament is affixable at an isometric distance to the joint pivot axis 208 in a medical procedure, using the AR headset. The plurality of points 202 in a circle (or cylinder) may be viewed as an overlay to the CT (computed tomography) scan and the location of the ring (or cylinder) is known in space. In one configuration, a single point may be shown that is at a distance of the isometric radius from the joint pivot axis 208.

Markers may be located on a femur and a patella to find isometric insertion points for the patella and femur. Alternatively, markers 210 may be placed on the skin of the leg but isometric insertion points may be defined based on where the bone is expected to be. FIG. 2 illustrates the marker 210 on the leg in a flexed position but the medical image shows the leg in extended position.

The joint pivot axis is where the center of the axis of rotation is for a joint and related ligament. The isometric point in the form of a circle or cylinder can be dragged out to be larger or smaller from the joint pivot axis based on the measured size for a ligament. For example, the desired ligament length may be equal to the radius of the circle, cylinder, cylinder segment, or arc. The circle or cylinder provides an isometric trajectory where an end of the ligament could be placed. In the case of FIG. 2, the size of the circle (or cylinder) may be adjusted to be bigger or smaller until the circle is on the patella. This circle then identifies where the isometric points for the patella are located. The other location where the second point of the ligament is to be placed can be found at the joint pivot axis of the femur or on the other relevant anatomy such as the surface of the bone. That area of the bone surrounding the joint pivot axis is where the drill hole may be placed to reach the joint pivot axis. Then the attachment point may be within the bone at the joint pivot axis.

In a more specific repair example, a procedure may be used to repair the patella in the knee. As the knee is flexed, the patella rotates forward across the ball of the distal femur. If this ligament has been torn, the medical procedure may re-anchor the patella to the femur. However, if the ligament is too short, then the ligament will rotate around the bone and stretch and tear. If the ligament is too long, then the ligament will be too loose and the patient may not have proper control of the anatomy. Accordingly, it is important to be able to attach a second end of the ligament to exactly that isometric hinge point which is an equal distance from the initial ligament attachment point at every point as the ligament moves.

More generally, there is a point of rotation where a joint rotates and a first end of a ligament may be attached at that location. This location varies based on anatomy of the structure, the persons size, any anatomic anomalies, etc. A hole may be bored into the bone to access the joint pivot axis and a new ligament or ligament replacement can be threaded into the hole. Unfortunately, the state of the art is to guess where to put both ends of the ligament. As discussed earlier, if the ligament is not isometric, then when the person moves the ligament will be too tight and the ligament will tear or the ligament will be too loose and the patient will not have proper control of the joint. Preferably, the ligament may be isometric across the hinge point of an elbow or knee. Otherwise, a ligament may stretch and be damaged.

It can be challenging to accurately identify a hinge point, such as a joint pivot axis or joint pivot point, prior to or during a medical procedure. This technology enables a medical professional to quickly and accurately identify a joint pivot axis or a joint pivot point during a medical procedure. The joint pivot axis or joint pivot point can be identified even without aligning and overlaying a 3D image data set over the anatomy of the person in the 3D space of the AR headset and without performing medical imaging (e.g., a CT (computed tomography) scan or an MRI (magnetic resonance imaging) scan) immediately prior to or during a surgery.

The medical professional may determine where the ligament is to be inserted on each side of the ligament, so as the joint moves through the range of motion, that distance of the ligament end point stays isometric to the hinge point. As discussed earlier, the marker may be placed on the distal anatomical structure or on both structures of a joint (e.g., fixed with a bone pin to the patella). Then the joint hinge axis may be computed and the joint hinge axis may be used as the first connection point for a ligament and the isometric points or isometric geometry may be used to find a second connection point. The motion discussed here is in one plane but the motion of the joint under consideration can be in any direction.

The system can calculate where the two ends of the ligaments may be more accurately placed. These locations are measured and calculated. The marker(s) allows the joint pivot axis to identified. The joint pivot axis 208 may be an axis line that passes perpendicularly through the image of FIG. 2, and the joint pivot axis 208 is the center axis of the knee. The ligament may need to be placed on the surface of a bone or as illustrated on the patella. The surgeon can virtually see where the bones are when a medical image data set (e.g., a CT scan or MRI) is aligned with the patient. As described, the arc, circle or cylinder centers on the joint pivot axis and can guide where the ligament should be placed based on measurements. According to the illustrated example of FIG. 2, one end of the ligament may be placed on the patella at one of the isometric points in the arc, circle or cylinder and the second end of the ligament may be placed at the joint pivot axis or hinge axis. The location where the patella is attached is a point that is isometric as defined by the arc, circle or cylinder.

This process can be used for other types of ligament reconstruction, such as an ACL reconstruction. For instance, the system can define where the normal attachment of the anterior cruciate ligament on the tibia is when a medical professional is coming through the bone to create a graft. The medical professional may be provided with a graphical guide at which to drill a hole. After the initial attachment point is created, then the isometric guide helps to identify where to attach the second end of the ligament. This technology can help with isometric ligament reconstruction and similar procedures.

As described, the AR headset may calculate a change between a first pose and a second pose. FIG. 2 illustrates that the angle between the first pose and the second pose may also be calculated and displayed as an arc 204 or an angle as an overlay using the AR headset. A numerical output 206 for the angle defining an angular change as the marker has moved around the joint pivot axis may also be displayed. In addition, the numerical angle 206 of the change between the first pose and the second pose may be displayed as an overlay as the angle changes in real time. More specifically, an angle of a displacement of the distal anatomical structure may be calculated using the first and second pose along with a graphical reference for the angular displacement displayed using the AR headset. This technology can track movement angles, the joint axis, distances, etc. using only the marker on the anatomy without needing any additional medical imaging.

In an example, the process may set a beginning point for measuring an angle. Initially, a marker 210 on the distal anatomical structure 212 may be detected. That location becomes the starting point for an angular measurement, then any movement from the first pose may be tracked and a report is provided about how far the marker 210 has moved from that starting point. A starting angle, displacement angle, and/or an ending angle may be displayed in the AR headset. Optionally, a marker on the body of the person or a proximal anatomical structure, can be used to get a relative angle measurement.

If a medical professional has the CT images open, then the medical professional may activate the slice view. In this example, the location where the axis crosses the slice of the CT scan can be seen as a point 208 on the angle of the rotation and arc segment. The circle or cylinder surrounds the joint pivot axis and shows where the ligament is to be anchored. As mentioned, the circle, ring 202 or cylinder may be called an isometric ring, isometric cylinder or isometric trajectory.

When the user is navigating through (e.g., deeper or shallower with respect to the view point, etc.) the anatomic slices of a CT scan or MRI scan that are perpendicular to the joint pivot axis, the joint pivot axis will appear throughout the slices. Normally, a medical professional is interested in viewing the bone in a ligament repair procedure. In contrast, if an ACL ligament procedure is being performed then the medical professional may desire to view deeper inside the knee using slices of the CT scan or MRI scan. When the medical professional finds the point for attaching the ACL, then the point may be marked.

Once the joint pivot axis 208 has been identified in a slice of an image data set, any point on the ring 202 is isometric from the joint pivot axis 208 because the ring 202 will be at the distance from the joint pivot axis 208 that is the length of the ligament to be re-attached (as long as the ring does not move). The ligament may be attached to the middle axis at any time. However, if the ligament is attached to a point on the isometric ring, the ligament needs to be at the same cross section of the axis as actually illustrated in the slice being viewed. Otherwise, a different distance may be used that is not in the slice (or in another slice of the CT scan) and the ligament may not be isometrically located.

Identifying and presenting isometric points may be extended to spherical hinges or ball joints like the hip and shoulder. When the joint pivot point is identified for a spherical hinge, a curved surface may be used to depict the isometric points.

Some joints are not a simple fit for illustrating isometric points, as in FIG. 2, but adjustments and representations may be provided in a graphical user interface to accommodate anatomical joint patterns or individual human variety. For example, in the knee, a joint pivot axis may move to a different point as you move through knee rotation. This technology may track where that joint pivot axis changes. The display of isometric points for joints where the joint pivot axis does not move may be less complex (such as the patella and tibia).

An example of using rotational displacement may be the example of a person who has broken their femur or hip in an accident. Not only has the bone broken but the bone has also rotated out of a normal position and around the longitudinal axis of the bone. The medical professional (e.g., a surgeon) wants to know how much to rotate the bone, so the bone and leg are returned to their correct or normal position. A CT scan of the leg may be obtained pre-operatively, and the medical professional can determine that the bone is displaced and/or rotated incorrectly and is resting at a certain angle. This angle is a measurable angle and the distance is measurable too. The displacement angle from a correct angle for the leg may be determined by comparing to the other leg. Suppose the leg bone has 30 degrees of rotational offset. With the known angle of rotation, a marker can be put on the end of the leg (i.e., the moving broken part). Then the AR headset can track this marker in the 3D coordinate space. A starting point may be set based on the current location of the bone, and then the bone and leg may be rotated through the measured 30 degrees. When the right point is reached, then the medical professional knows the procedure is complete. After the initial scan (e.g., CT scan or MRI scan) and measurement, no other imaging is needed for such a procedure. In addition, this process does not need a navigation system or need to be registered to a sensor array, etc.

In another example of rotational displacement, the anatomical structure may have rotated 60 degrees but the bone may have also moved or translated in position away from the other part of the broken bone. The system can keep track of the displacement in space and the rotational angle of displacement. A bone may have been translated 8 cm (centimeters) and rotated 60 degrees. Multiple points on the marker are tracked, and tracking many points on the marker allows for 6 degrees of freedom of orientation. This detailed tracking means there is only one rotation and translation around a single axis that can create the rotation using a plurality of points on the marker (e.g., optical code). The rotation and displacement of an anatomical structure (the leg, foot, arm, etc.) may be displayed. The user may be instructed to move the anatomical structure 8 cm while using a 60 degree rotation. As described, the axis of rotation may down an axis of symmetry of the anatomical structure (e.g., down the length of the leg, arm or bone). When the rotation and translation is being made, the displacement or translation in space may be reported to a medical professional or user.

An image data set can also be aligned to the body of the person using the marker. Medical imaging may be obtained and aligned with a body of a person. For example, a CT (computed tomography) scan, MRI (magnetic resonance imaging) image or other imaging may be overlaid on the patient and used to obtain measurements of anatomical aspects of joint or limb being operated on. U.S. Pat. Nos. 9,892,564; 10,475,244; 11,004,271; 10.010.379:10,945,807; 11,266,480; 10,825,563:11,237,627; 11,287,874; U.S. patent application Ser. No. 17/706,462 entitled “Using Optical Codes with Augmented Reality Displays”; and U.S. patent application Ser. No. 17/536,009 entitled “Image Data Set Alignment for an AR Headset Using Anatomic Structures and Data Fitting”; and U.S. patent application Ser. No. 17/978,962 entitled “3D Spatial Mapping in a 3D Coordinate System of an AR Headset Using 2D Images” describe methods and systems for aligning an image data set from medical imaging devices with a body of a person and these descriptions are incorporated in their entirety by reference herein.

In another configuration, the markers may be detected and may be used for moving a portion of an anatomy, such as a bone, that is in dislocated or broken position to a correct location. To perform this function, a target position and rotation may be set to which the marker is desired to be moved in the 3D space. Next, a positional distance and a rotational angle of a displacement of the distal anatomical structure may be tracked from a start point using the marker. A rotation axis may be defined that is a longitudinal axis of a bone in the distal anatomical structure or the proximal anatomical structure. Then a graphical reference may be displayed for positional distance and rotational angle of displacement as the distal anatomical structure is moved and rotated from the start point. A graphical message may also be displayed when the target position and rotation has been reached. For example, the distal anatomical structure and proximal anatomical structure may be parts of a broken bone, a dislocated joint, or dislocated anatomical structure.

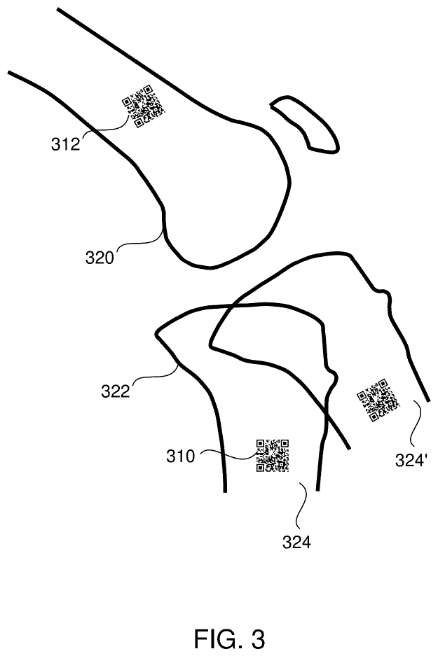

FIG. 3 illustrates the use of a first marker 310 on a distal anatomical structure 322 of the joint and a second marker 312 on a proximal anatomical structure 320 of the joint. The distal anatomical structure 322 and the proximal anatomical structure 320 may be referenced to each other rather than the 3D space of the AR headset.

A first pose position for the second marker 312 in a 3D (three dimensional) space may be registered. Then a target position and rotation for the second marker 312 may be set in the 3D space. The positional distance and a rotational angle of a displacement of the distal anatomical structure 322 may be computed using the first pose position 324 and second pose position 324′. The positional distance and rotational angle as the distal anatomical structure is moved may also be displayed between the first pose position and the second pose position, using the AR headset. Visual, audible, tactile (e.g., vibrations) or graphical feedback can be provided when the target position and location have been reached. For example, a graphical icon may be displayed when the target position or rotation is reached.

The first marker 310 and distal anatomical structure 322 may be related to anatomical structures that are not joints. In one configuration, the distal anatomical structure and the proximal anatomical structure may be related to a broken portion of a bone.

Structures of anatomy related to the joint may also be segmented using machine learning, machine vision, edge detection and similar processes. The segments of anatomy may be moved, as viewed through the AR headset, using changes in position and orientation of one or more markers. For example, the system may segment the different bones related to a joint or the person's anatomy. The segments may be virtually moved with the marker that is being moved and viewed in the AR headset.

A joint pivot point of a ball joint for a patient may also be identified and displayed using this technology. A type of ball joint for which a joint pivot point may be determined may include a hip joint or a shoulder joint. A joint axis point in the ball joint may be determined by taking multiple pairs of poses, and each pair of poses can be used to find the joint pivot axis and angle for the pair of poses. The multiple pivot axes may be intersected and this will identify a single point that is ball joint's pivot point.

More specific details of this joint pivot point finding process may be that a marker in a first pose on an anatomical structure forming the joint with proximal anatomical structure may be registered. A second pose of the marker after the anatomical structure has been moved in a first plane with respect to the proximal anatomical structure may then be registered. A first joint pivot axis may be registered by identifying an intersection of a first line that is parallel to a plane created by points of the first marker and a second line that is parallel to a plane created by points of the second marker. The distal anatomical structure (e.g., leg or arm) may be then be moved to a second plane of movement and a third pose and fourth pose with the marker may be registered to identify a second joint pivot axis. An intersection of a first joint pivot axis and the second joint pivot axis for the joint pivot point for the person's ball joint may then be found or computed.

An image data set may also be aligned to a person using the first marker. The joint pivot point and a plurality of points in a curved surface surrounding the joint pivot point that are isometric to the joint pivot point, and the image data set may be displayed using the AR headset. The plurality of points in the curved surface may be used for determining isometric points for attaching a ligament.

The present technology is convenient because it may be used with markers (e.g., visible or optical markers) and this avoids the use of medical images with the rotation and alignment. Orthopedic medical professionals move bones frequently and this system can assist with orthopedic procedures. The ability to track anatomical structure movement moved by a medical professional's hand in real time while the bones are moving is valuable because it avoids the use of external machinery, protractors, compasses, etc. A medical professional may also receive display information about the angle of movement, the joint pivot axis, or an isometric circle or cylinder for the joint pivot axis which can be helpful in such procedures.

Medical professionals may also perform these procedures with AR but without pre-planning. For example, a rotation of a broken bone may not need to be planned in advance. The medical professional may decide to rotate the femur 30 degrees for therapeutic purposes but the medical professional does not need to perform imaging during the medical procedure because this system can identify such angles and changes in position during the medical procedure.

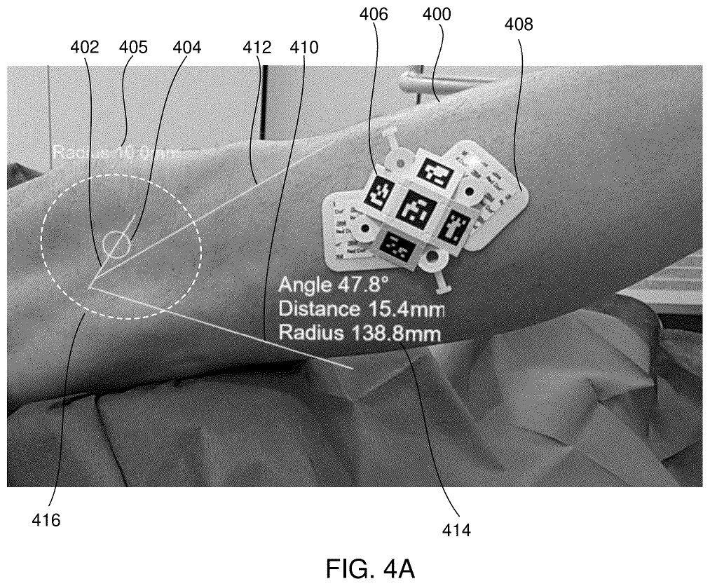

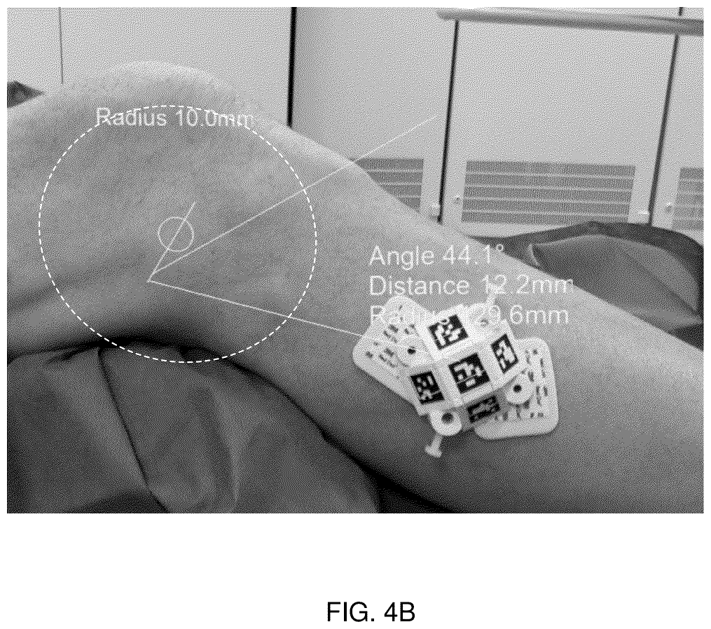

FIG. 4A illustrates an example of tracking the tibia in a leg in extension and identifying a joint pivot axis 402 or hinge point of a knee joint of the leg 400 between the tibia and femur. A circle 404 (or slice of a cylinder) or an entire cylinder may be used with an isometric radius that defines where one end of a ligament may be attached on a bone (e.g., the patella) and the center of the circle may define where another end of the ligament may be attached (e.g., on the femur). The circle or cylinder may start out at a default size 405 and then the user may drag the circle 416 or cylinder out to increase the circle's size to the circle 416 or cylinder that covers or depicts locations where the ligament may be isometrically attached to the patella. The numerical size of the isometric radius of the circle 416 or cylinder may also be displayed (e.g., the default radius may be 10 mm and the expanded radius may be larger). The joint pivot axis 402 depicted in FIG. 4A is illustrated as being viewed from a right side and at a perspective angle from the joint pivot axis 402 as the joint pivot axis 402 extends in 3D space away from the AR headset viewer.

A marker that includes an optical code 406 (and more specifically includes a plurality of a 2D optical codes) can be attached to the leg 400, for example, using a bone pin, a wrap, adhesive layers 408 or adhesive pads and a connective marker structure. The marker 406 may be used to identify the movement of the tibia with respect to the femur. The movement of the tibia may be tracked between a first position, as marked by a first line 410, and a second position as marked by a second line 412. The intersection of the first line 410 and second line 412 may be used to identify the joint pivot axis 402. The first line 410 and second line 412 may be located at the skin of the limb, along a surface of a bone or at a center of a bone. The angle of displacement or movement (e.g., 47.8 degrees) between the first position and second position may be displayed 414 as an overlay in the AR headset, along with the distance of a sideways translation motion (e.g., 15.4 mm (millimeters)) during movement from the first position to a second position and a length the structure (i.e., leg) from the joint pivot axis to the marker (e.g., 138.55 mm)). FIG. 4B illustrates an example of tracking the tibia in a leg (also shown in FIG. 4A) in flexion and identifying a joint pivot axis of a knee joint between the tibia and femur.

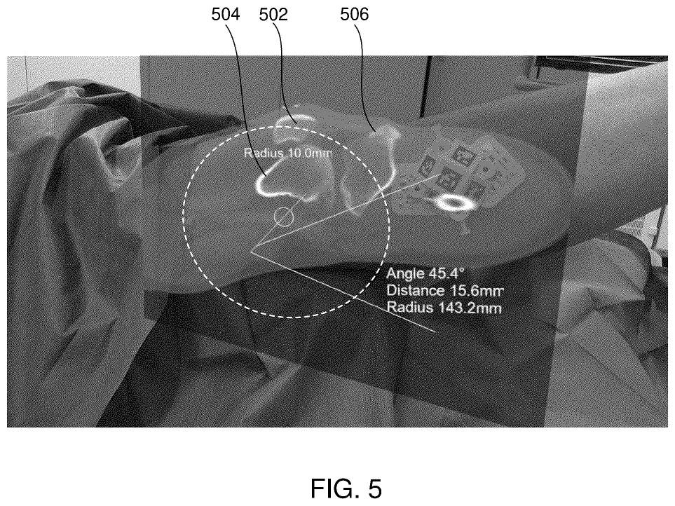

FIG. 5 illustrates an example of tracking a tibia 506 in a leg in extension and identifying a joint pivot axis of a knee joint between the tibia and femur as in FIGS. 4A and 4B. In addition, FIG. 5 illustrates that an image data set from a medical imaging device may be overlaid on the leg. For example, the image data set may be a CT scan, MRI, x-ray based image, or another type of medical image. The image data set may provide a view of a specific slice of the image data set. In the example of FIG. 5, a cross section slice of a patella 502, femur 504 and tibia 506 may be seen.

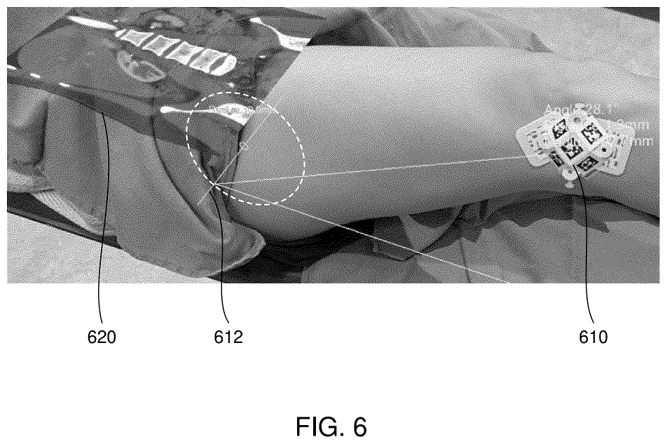

FIG. 6 is an illustration of a marker 610 affixed to a leg (e.g., at or near the bottom of the femur). The leg may then be moved to various different poses (e.g., two or more) with the marker 610 and then one joint pivot axis 612 of the joint (one axis at the top of the femoral head) may be identified. The length of the femur may be also be identified by providing the ability to measure from the joint pivot axis to the end of the femur using an image data set overlaid using the AR headset. Alternatively, the distance from the joint pivot axis 612 to a marker may be measured. A 2D (two dimensional) image data set representing the pelvis (not the hip being discussed) is depicted to illustrate the ability to include aligned and overlaid images but image data sets of the hip joint may also be used as an overlay in the AR headset.

A joint pivot point 612 for a ball joint may also be determined by using two groups of poses in at least two pose planes for the leg. For example, two poses may be captured in plane A which can enable to the joint pivot axis 612 to be identified. Then two more poses may be identified in plane B (not shown) and a second joint pivot axis (not shown) may be identified. The intersection of the joint pivot axis for plane A and plane B can then be used to identify the joint pivot point for the leg's ball joint.

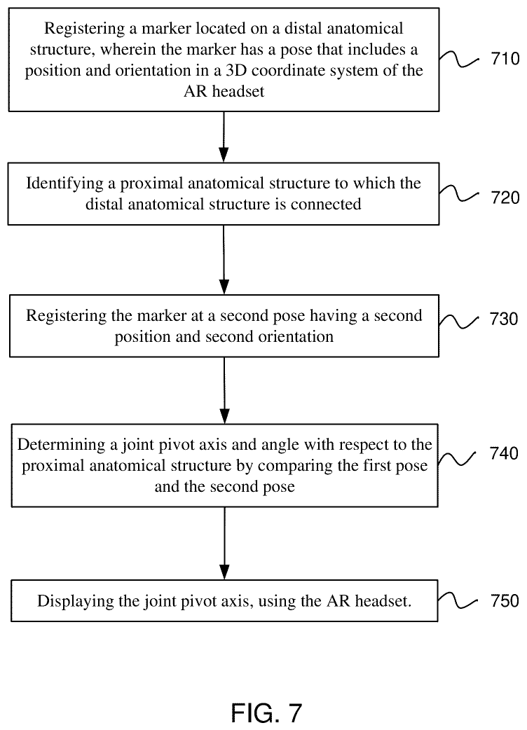

FIG. 7 is a flowchart illustrating an example method for referencing anatomical structure using an AR headset. The method may include registering a marker located on a movable anatomical structure, as in block 710. The marker may have a first pose that includes a position and orientation in a 3D coordinate system of the AR headset.

A proximal anatomical structure may be identified to which the movable anatomical structure is connected, as in block 720. In some situations, the movable anatomical structure may also be considered a distal anatomical structure. The proximal anatomical structure may be the structure that is not moving substantially during the use of the AR headset or is substantially fixed in place due to restraints or body weight. The marker may be detected at a second pose of the movable anatomical structure having a second position and second orientation, as in block 730.

A joint pivot axis and angle may be determined by comparing the first pose and the second pose, as in block 740. The joint pivot axis may then be displayed using the AR headset, as in block 750. In one example, the proximal anatomical structure may include a bone and the movable anatomical structure (or distal anatomical structure) may be a patella.

In another configuration a second marker having a third position and third orientation on the proximal anatomical structure may be registered. The first pose and second pose may then be computed with respect to the proximal anatomical structure using the third marker to enable free movement of the movable anatomical structure (e.g., distal anatomical structure) and the proximal anatomical structure. The markers may be at least one of: a visible marker, an optical code, a linear bar code, a 2D bar code, an infrared marker, or a radiopaque marker.

A plurality of points may be displayed surrounding the joint pivot axis that are isometric to the joint pivot axis. The points may form curves, circles or other geometric shapes or surfaces. For example, the plurality of points may form at least one of: an arc, a circle, a cylinder, a curved surface, a sphere, a smooth surface, or an irregular shape. A plurality of isometric points may be displayed surrounding the joint pivot axis based on a defined ligament length to identify locations on a bone where a ligament is affixable at isometric distances to the joint pivot axis in a medical procedure.

A length of a bone or portion of a bone can also be measured from the joint pivot axis to a point defined on the bone. An image data set aligned to the person using the marker may be used to more accurately measure the length of bones or measure between the joint pivot axis and points on the bones. In one example use case, an anatomical landmark may be marked in femur and the medical professional can make a leg length measurement from the landmark to an end of the bone.

Changes in an angle between the first pose and the second pose may also be calculated. Then a numerical output may be displayed for the angle defining an angular change as the marker moves around the joint pivot axis.

A graphical guide may be provided to guide surgical access to the joint pivot axis or a plurality of isometric points on a bone that are related to the joint. This may include graphical images or guidance icons to guide a medical professional to the joint pivot axis or the isometric points on the bone (e.g., a drilling path, a scalpel path, etc.). For example, the graphical guide may illustrate a graphical shape on the bone that indicates where to drill or a channel to use to drill into the bone. The graphical guide may be displayed to the user through the AR headset.

This technology may be used with any type of mixed reality device. Examples of mixed reality devices with which this technology may be used may include: VR (Virtual Reality) devices, flat panel displays using augmented cameras, head-up displays or other systems where the interfaces of the technology may be displayed along with real-time views or real-time video images of patient.

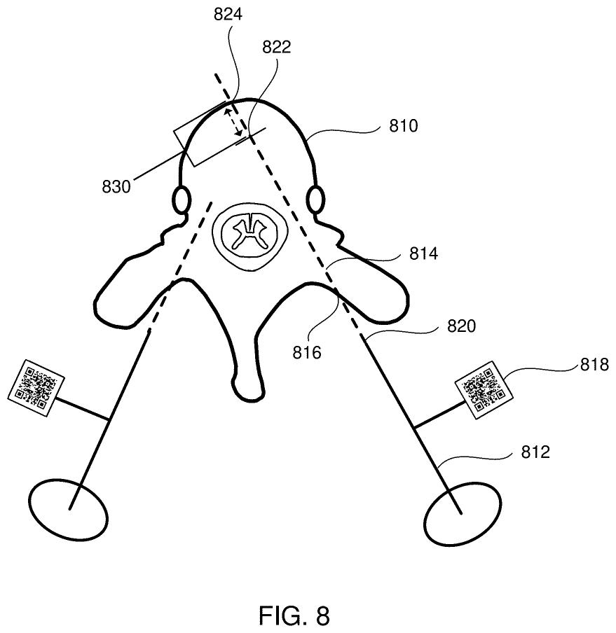

FIG. 8 illustrates a system and method for referencing anatomical distance or automatic measurements for medical hardware (e.g., screw or implant) using an augmented reality (AR) headset. More specifically, the technology may use a pointer device to automatically find an anatomic path, an anatomic length, or a medical implant length, width or height to determine an anatomical path and structure during a medical procedure. An initial operation may be determining at least one boundary of an anatomical structure for a body of a person. An example boundary of the anatomical structure may a boundary of a bone 810 or shape of a bone, such as a vertebra. The boundary may include the entire 3D volume, a 2D shape of an anatomical structure displayed in an image data set or just one or more portions of the boundary of the anatomical structure.

An image data set from a medical imaging device (e.g. CT scan or MRI) may be aligned to the body of the person using a marker on the body of the person or on the anatomical structure. The marker may be an optical code or another type of marker as referenced in this description. The image data set may be viewed through an AR headset so that the image data set appears as a graphical overlay on top of the real view of the person through the AR headset. The image data set may be used by an anatomical structure detection service to detect the boundaries of an anatomical structure(s) in the image data set. The boundaries of a specific anatomical structure may be identified using edge detection, feature detection, shape detection, morphometric detection, machine learning or other shape or volume detection processes that may be applied to the image data set.

A pointer device 812 may be registered with the AR headset and the pointer device 812 may have a position and orientation with respect to the 3D coordinate system of the AR headset or the anatomical structure 810. A marker 818, such as an optical code connected to the pointer device, may enable the AR headset to register the position and orientation of the pointer device.

A virtual line 814 may be generated that has a lengthwise axis aligned with a lengthwise axis of the pointer device 812 and the virtual line 814 may pass through a portion of the anatomical structure. For example, the virtual line may extend from a tip 820 of the pointer device 812 and away from the pointer device in one (or two directions). A medical professional may direct the tip of the pointer device 812 toward the anatomical structure while the virtual line 814 is generated. While the virtual line 814 is shown as dotted, the virtual line may be solid (not dotted), semi-one dimensional (e.g., one physical display element in width or height), 2 dimensional (e.g., having limited width) or three dimensional (e.g., having a limited height and width).

A measurement may be determined from an entry point 816 where the virtual line 814 enters a boundary of the anatomical structure 810 to an additional point 822 in the anatomical structure. The AR headset may display a numerical output for a length of the virtual line between the entry point 816 where the virtual line enters the anatomical structure to the additional point 822 defined for the anatomical structure. The length of the virtual line 814 may be used to determine a length of a medical device to be used with the anatomical structure based on the length of the virtual line 814 between the entry point 816 where the virtual line enters the anatomical structure to an additional point in the anatomical structure. For example, the medical device may be at least one of an implant, a screw, a needle, a stent or a trocar.

The additional point may be a point defined by a user within the anatomical structure anywhere along the virtual line. In another example, the additional point may be where the virtual line exits from the anatomical structure. Alternatively, the additional point on the line may be a set distance computed from the entry point 816. For example, the line may extend 5 cm from the entry point 816.

In another configuration, the additional point may be a location along the virtual line that is determined using a distance offset 830 internal from an exit location 824 where the virtual line may exit the anatomical structure. For example, the exit location 824 of the virtual line may be identified and then an offset location on the virtual line 814 may be computed that is 1 cm (centimeter) internal from the exit location (or what would otherwise be the exit location if the virtual line 814 is shorter). Then the length of the virtual line 814 may be measured from the entry location 816 to the offset location that forms the additional point 822.

The measured distance can be used to determine the length of a medical tool (e.g., screw or rod) that may be used in the part of the anatomy that the line is overlaid upon. In an example use of this measurement technology illustrated in FIG. 8, a needle or instrument can be used as pointer device 812 and a marker 818 may be located on or attached to the device. As a medical professional guides the pointer device 812 toward the anatomical structure, the image data set can be reconstructed to display different planes relative to the tip of the instrument. The plane or slice of the image data set being viewed may be controlled by the location of the needle with respect to the anatomical structure (e.g. show the layer closest to the coordinate of the pointer device tip). Alternatively, the plane or slice of the image data set being viewed may be controlled by the medical professional wearing the AR headset.

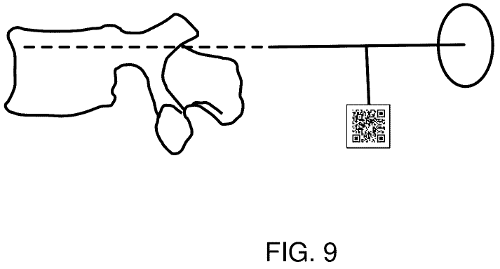

FIG. 8 illustrates an axial view of vertebra with a needle and a projected virtual line or projected ray from the top of the needle. FIG. 9 illustrates a sagittal view of the vertebra and needle of FIG. 8. The AR headset may provide smaller inset orthogonal views of the image data set and pointer device in the AR headset. The image data set may be viewed as a MIP (maximum intensity projection) view, a direct volume rendered image or another view of the 3D image data set.

When the virtual line is projected from a tip of the pointing device or needle, the virtual line may intersect a first part of anatomical structure (e.g., the bone), then this point may be automatically recorded or the medical professional may mark the entry point. The AR headset may be tracking the needle in three dimensions. As discussed earlier, the image data set may be aligned to and overlaid on the patient through the AR headset and the image data set enables the measurements to take place. The aligned image data set may also be used to help determine when the virtual line has intersected with the anatomical structure in the 3D coordinate system of the AR headset. An intersection with the virtual line and any boundaries of the identified anatomical structures identified from the image data set can be computed.

In a further example, a virtual screw of a defined length may be created to see if the virtual screw can fit in the anatomical structure (e.g., bone) that is identified. If a screw is too long then the screw might extend out the other end of the vertebral body and kill the patient (e.g., the screw might hit the aorta). As a result, the virtual screw may be sized to a safe length or a desired length by using the virtual line and the pointer device. Further, the system may suggest implant or screw sizes based on the detected boundary of the anatomical structure and/or the line within the anatomical structure. The system may also identify an exit boundary for the virtual line that is crossing the bone boundary and compute a safety margin for the screw. This allows the length of a desired screw to be calculated based on the entry point of the virtual line into the anatomical structure and an end point in the bone that has a safety distance or safety factor accounted for in the length of the screw. Further, the virtual sizing may occur in any direction or dimension such as length, width or height. For example, a diameter of a screw may be determined to see if the screw may breach a narrow bone (e.g., a narrow pedicle of the spine).

This technology may also be used for automatic depth measurement. If a medical professional is going to perform a tap for a fluid collection, then knowing the depth of the fluid filled area is valuable. The medical professional may point the pointer device or instrument at the area of anatomical structure where the fluid is expected to be, then the virtual line may point to the area with fluid. This may allow the medical professional to determine the depth to which a needle or trocar needs to travel to hit the fluid area. In addition, this technology may allow the medical professional to measure an anatomical structure for a stent and determine a length of stent prior to putting the stent into the patient.

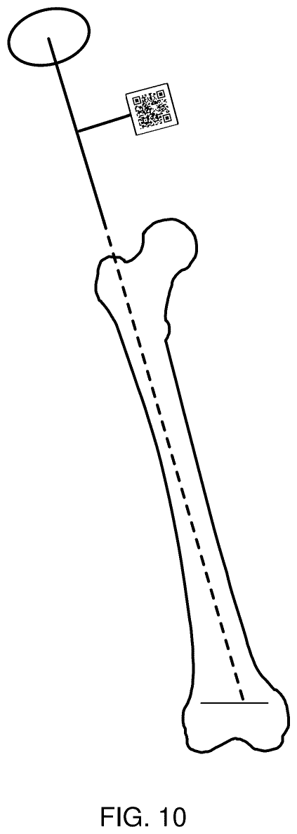

FIG. 10 illustrates measuring an anatomical distance for use in repairing a fracture in a femur. The virtual line from a pointing device may be used identify a length of a portion of a bone where a nail or screw is to be implanted to bridge across a fracture in the broken femur. It is useful to know the needed length of the screw or nail in advance so that when the screw or nail is placed, the implant is not too long or too short. The virtual line used with the pointer device may assist in finding the appropriate location and then measuring the desired path in the bone. A margin of safety can also be determined during the measuring operation, as described earlier.

When an augmented reality (AR) headset is used with medical procedures, it can be cumbersome to do planning with the headset on or during the procedure because the medical professional does not have a mouse or a workstation nearby. In addition, the images may change or move around based on the medical professional's perspective. This technology may give the doctor the ability to be able to instrument or measure something without interrupting or leaving a procedure.

Another example of using this technology is working to place a ventricular catheter in the brain. The generation of the virtual line can be used to find the distance to a ventricle. The technology can automatically find the ventricles in a CT scan and then measurement may take place. Measurements through an AR headset are useful because sometimes a medical professional does not have the time to perform measurements due to receiving an emergency patient or the medical professional has limited time based on insurance time and billing limitations.

There may be situations in medical procedures where a medical professional may want to align an image data set (e.g., a CT scan or MRI scan) with a body of a person or patient to perform a medical procedure. However, the image data set may represent a different scanned position for the person's anatomical structure than the desired position of the anatomical structure in which the medical procedure is to be performed. For example, in the case of a ligament insertion into a joint, the scans for the image data set may have been performed in the medical imaging device when the joint (e.g., leg, elbow, knee, etc.) is extended or straight. However, if endoscopic types of medical tools are to be used on the joint (e.g., the knee) during the medical procedure, then the medical professional may need to bend the knee significantly (e.g., to approximately 90 degrees) to insert the endoscopic tools. Further, a person's anatomy cannot be significantly bent in the medical imaging device (e.g., CT scanner or MRI scanner) due to lack of space in a medical imaging device, and the scanner itself cannot be bent. Essentially, there is practically no room in the medical scanner to bend body parts in any significant amount during medical image scanning.

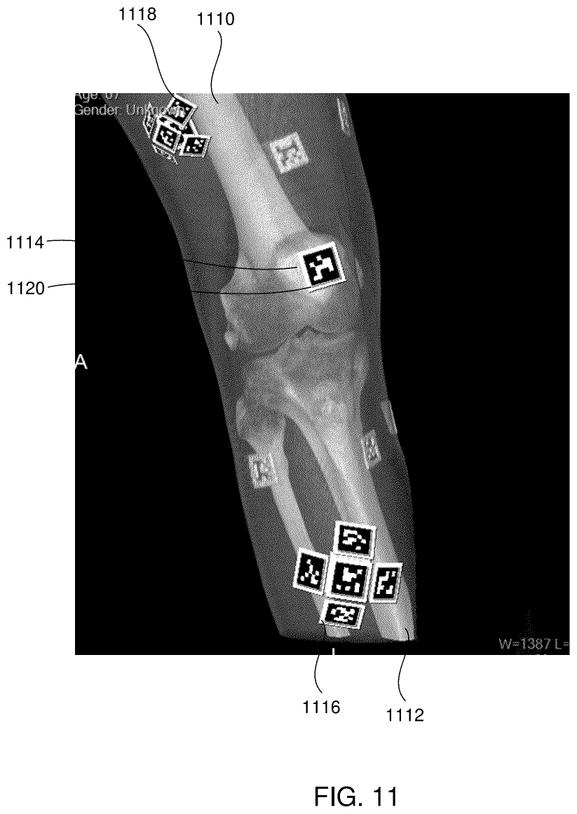

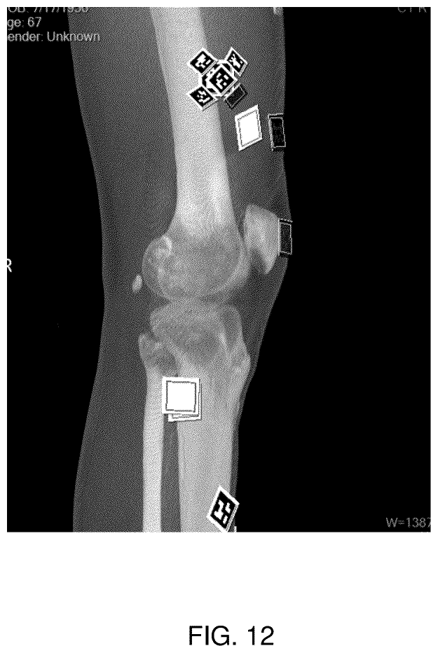

The present technology provides the ability for virtual repositioning of anatomical structure in an image data set. Then the revised image data set may be displayed in an AR headset and aligned with a person or patient. The image data set of FIG. 11 illustrates a 3D scan of a knee in an extended position with markers 1116, 1118, 1120 (e.g., using 2D optical codes) on the femur 1110, tibia 1112 and patella 1114. The 3D scan may be a 3D CT scan or an MRI scan. FIG. 12 is a sagittal view of a knee in an extended or unbent pose.

The image data set may be aligned to the person using the marker (e.g., 2D optical code or another marker) associated with the person or on the person. A marker located on a movable anatomical structure of a person may be registered with the AR headset. The marker may then be detected at a first pose that includes a position and orientation in a 3D coordinate system of the AR headset.

A joint pivot axis and angle may be determined by comparing a first pose and a second pose of the movable anatomical structure using the marker. For example, the location and orientation of the marker may be detected when a distal part of the joint is moved by a person or medical professional to a first pose and a second pose.

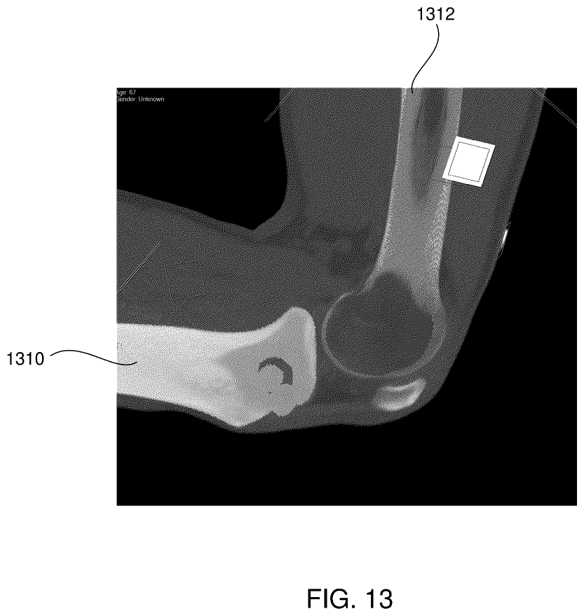

The structures of anatomy in the image data set may be segmented to form anatomical segments. In one example, the structures of anatomy may be related to a joint of the person. FIG. 13 illustrates bone segments 1310, 1312 that are identified near a joint of a person. In a further example, the boundaries of bones, organs, blood vessels, or anatomic structures may be identified in the image data set. The segmenting of structures of anatomy may include detecting bones and anatomical structure using edge detection, feature detection, shape detection, or machine learning.

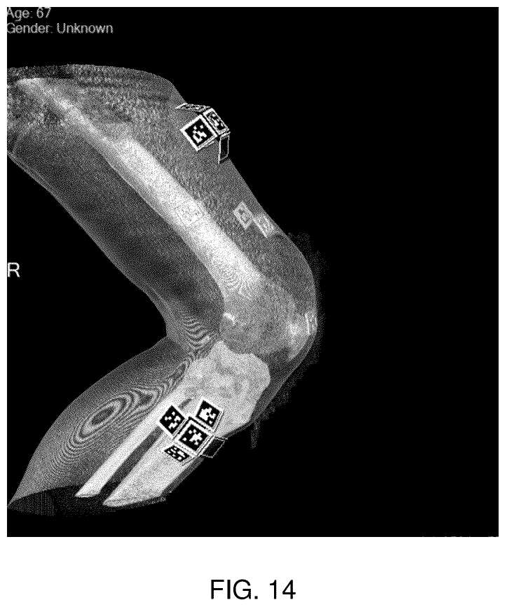

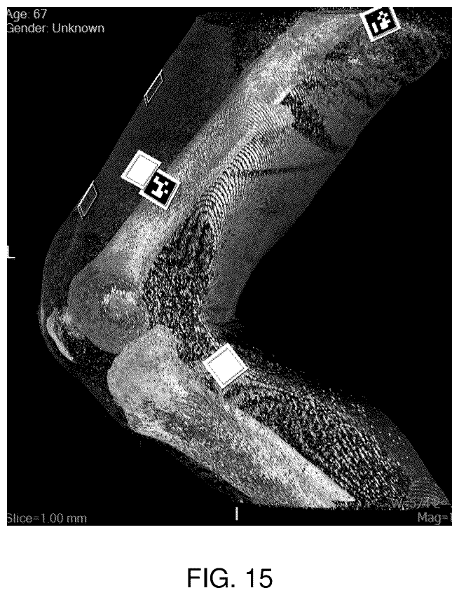

FIGS. 14 and 15 illustrate that the anatomical segments may be rotated around the joint pivot axis in the image data set. FIG. 14 may be an example of rotating anatomical segments from the image data set of FIG. 11. The segments identified may be rotated around the joint pivot axis to represent flexion in the image data set and represent the movable anatomical structure of the person. For example, the anatomical segments may be rotated around the joint pivot axis to an angle that matches an angle of flexion for a real joint viewable through and detectable by the AR headset. If the angle of a patient's visible joint is 45 degrees as viewed through and detected by the AR headset, then the anatomical segments may be rotated 45 degrees. The anatomical segments may be virtually moved and/or rotated in the 3D image space of the image data set. When the image data set is aligned with the person, then the moved anatomical segments in the modified image data set may appear to match the person's visible anatomical pose. More specifically, the modified image data set may be viewed using the AR headset. In another example, the bones may be segmented and then the bones can be virtually rotated using an application, service or process executed using the AR tracker to perform the processes described earlier.

In the past, bones and anatomical structures could be found and segmented in an image data set using AI and other segmentation techniques. However, there has not previously been a way to make changes to an image data set to model changes to a person's anatomy (e.g., bending of a joint) after the image data set has been captured by a medical imaging device. In addition, there has not been an ability to move the segmented bones and anatomical structure in a way that was helpful in a medical procedure. Using this technology, the movement and paths of bones and anatomical structures can be predicted and then applied to the image data set. More specifically, this technology can identify the joint pivot axis and use that to compute the correct rotational movement of bones and anatomical structures around the joint pivot axis. These changes can be applied to an image data set without capturing an additional image data set using a medical imaging machine. For instance, the changes may be applied during a medical procedure, as mentioned earlier.

In the example of a leg, a femur and a tibia may be segmented from the image data set with the knee joint in extended position. Then the tibia can be virtually and correctly rotated around the femur in the image data set using the joint pivot axis found using markers on a person's joint and the AR headset, as described earlier. The insertion point for a ligament that may be used for planning the medical procedure is still valid in this scenario. Upon segmentation using AI, the bone and other identified tissue (e.g., skin, blood vessels, organs, etc.) can be pivoted around the joint pivot axis or virtual hinge point. This allows the medical professional to maintain the relative alignment of the two bones even though the bones and anatomical structures were not scanned in that position. The joint pivot axis or other information (e.g., skin location, etc.) obtained from using a marker while moving the leg (e.g., finding the joint pivot axis) can be used to virtually move and/or associate two independent structures (e.g., bones) correctly in a joint.

It can be difficult to model the soft tissue because the soft tissues stretches or bends. The tissue proximal to the joint may be deformed or stretch more when a joint is bent, but soft tissue more distal to the joint may be modeled more predictably because of reduced deformation. Over time accurate models may be created of soft tissue and how the soft tissue deforms during bending or other movement cases. Then these soft tissue deformation models may also be applied during the movement of the segmented anatomical structure.

Some of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices. The modules may be passive or active, including agents operable to perform desired functions.

The technology described here can also be stored on a computer readable storage medium that includes volatile and non-volatile, removable and non-removable media implemented with any technology for the storage of information such as computer readable instructions, data structures, program modules, or other data. Computer readable storage media include, but is not limited to, RAM, ROM, EEPROM, flash memory or other memory technology, optical disks, or other optical storage, magnetic cassettes, magnetic tapes, magnetic disk storage or other magnetic storage devices, or any other computer storage medium which can be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared, and other wireless media. The term computer readable media as used herein includes communication media.

Furthermore, the described features, structures, or characteristics may be combined in any suitable manner in one or more examples. In the preceding description, numerous specific details were provided, such as examples of various configurations to provide a thorough understanding of examples of the described technology. One skilled in the relevant art will recognize, however, that the technology can be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements can be devised without departing from the spirit and scope of the described technology.

What is claimed is:

1. A method for referencing anatomical structure using an AR headset, comprising:
registering a marker located on a movable anatomical structure, wherein the marker has a first pose that includes a position and orientation in a 3D coordinate system of the AR headset;
identifying the marker at a second pose of the movable anatomical structure having a second position and second orientation; and
determining a joint pivot axis and angle by comparing the first pose and the second pose.

2. The method as in claim 1, further comprising displaying the joint pivot axis, using the AR headset.

3. The method as in claim 1, further comprising:
registering a second marker having a third position and third orientation on a proximal anatomical structure at a second pose; and
computing the first pose and second pose with respect to the proximal anatomical structure using the second marker to enable free movement of the movable anatomical structure and the proximal anatomical structure.

4. The method as in claim 1, further comprising displaying a plurality of points surrounding the joint pivot axis that are isometric to the joint pivot axis.

5. The method as in claim 4, wherein the plurality of points forms at least one of: an arc, a circle, a cylinder, a curved surface, or an irregular shape.

6. The method as in claim 1, further comprising displaying a plurality of isometric points surrounding the joint pivot axis based on a defined ligament length to identify locations on a bone where a ligament is affixable at isometric distances to the joint pivot axis, using the AR headset.

7. The method as in claim 1, measuring a length of a bone from the joint pivot axis to a point defined on the bone.

8. The method as in claim 1, further comprising:
calculating a change in the angle between the first pose and the second pose; and
displaying a numerical output for the angle defining an angular change based in part on movement of the marker around the joint pivot axis.

9. The method as in claim 1, further comprising aligning an image data set to a person using the marker attached to the person.

10. The method as in claim 1, further comprising identifying the joint pivot axis as an intersection of a first line that extends down a surface of a bone of a joint substantially parallel to the first pose of the marker and a second line that extends along a surface of the bone of the anatomic structure at the second pose.

11. The method as in claim 1, wherein the movable anatomical structure and a proximal anatomical structure include bones forming a joint.

12. The method as in claim 1, wherein a proximal anatomical structure includes a bone.

13. The method as in claim 1, further comprising providing a graphical guide to guide surgical access to the joint pivot axis or a plurality of isometric points on a bone that is related to a joint.

14. The method as in claim 1, wherein the marker is at least one of: an optical code, a 2D bar code, an infrared marker, or a radiopaque marker.

15. The method as in claim 1, further comprising identifying a proximal anatomical structure, which is fixed in the 3D coordinate system, and the movable anatomical structure is connected to the proximal anatomical structure.

16. The method as in claim 15, further comprising determining the joint pivot axis and angle with respect to the proximal anatomical structure by comparing the first pose and the second pose.

17. A method for referencing anatomical structure of a person using an AR headset, comprising:
registering a marker on a distal anatomical structure, which is connected with a proximal anatomical structure that does not move during a medical procedure;
identifying a first pose for the marker in a 3D (three dimensional) coordinate system and a second pose for the marker;
calculating a rotational angle of a displacement of the distal anatomical structure using the first pose and second pose; and
displaying a graphical reference for the rotational angle of displacement, using the AR headset.

18. The method as in claim 17, further comprising:
setting a target position and rotation for the marker in the 3D coordinate system;
tracking a positional distance and the rotational angle of a displacement of the distal anatomical structure from a start point using the marker; and
displaying the graphical reference for positional distance and rotational angle of displacement as the distal anatomical structure is moved and rotated from the start point.

19. The method as in claim 18, further comprising providing at least one of a visual, audible or tactile indicator when the target position and rotation for the marker has been reached.

20. The method as in claim 17, wherein the distal anatomical structure and the proximal anatomical structure are parts of at least one of: a broken bone, a malrotated structure, a dislocated joint, or dislocated anatomical structure.

21. A method for referencing anatomical structures for a joint of a person using an AR headset, comprising:
registering a first marker on a proximal anatomical structure of the joint and a second marker on a distal anatomical structure of the joint;
identifying a first pose position for the second marker in a 3D (three dimensional) space;
setting a target position and rotation for the second marker in the 3D space;
identifying a second pose position for the second marker in a 3D space;
computing a positional distance and a rotational angle of a displacement of the distal anatomical structure using the first pose position and second pose position; and
displaying the positional distance and rotational angle as the distal anatomical structure is moved between the first pose position and the second pose position, using the AR headset.

22. The method as in claim 21, further comprising aligning an image data set to the person using the first marker or the second marker attached to the person.

23. The method as in claim 21, providing visual, audible, tactile, or graphical feedback when the target position and location have been reached.

24. The method as in claim 23, further comprising displaying a graphical icon when the target position or rotation is reached.

25. The method as in claim 21, further comprising:
segmenting structures of anatomy related to the joint to form segments; and
moving the segments, as viewed through the AR headset, using changes in position and orientation of the first marker or the second marker.

26. The method as in claim 21, wherein the proximal anatomical structure and the distal anatomical structure include a broken portion of a bone.

27. The method as in claim 21, further comprising defining a rotation axis that is a longitudinal axis of a bone in the proximal anatomical structure or the distal anatomical structure.

28. The method as in claim 21, wherein the first marker and the second marker are at least one of: an optical code, 2D optical code, an infrared marker, or a radiopaque marker.

29. The method as in claim 21, further comprising registering a plurality of points on an optical code.

30. The method as in claim 21, wherein the proximal anatomical structure is fixed in place within the 3D space.

31. A method for identifying anatomical structures for a ball joint of a person using an AR headset, comprising:
registering a marker in a first pose on a distal anatomical structure which is connected with a proximal anatomical structure;
registering a second pose of the marker after the distal anatomical structure has been moved with respect to the proximal anatomical structure;
determining a first joint pivot axis and angle with respect to the proximal anatomical structure by comparing the first pose and the second pose;
registering a third pose and fourth pose with the marker to identify a second joint pivot axis; and
determining an intersection of a first joint pivot axis and the second joint pivot axis that represents a joint pivot point of the ball joint.

32. The method as in claim 31, further comprising displaying the joint pivot point using the AR headset.

33. The method as in claim 31, further comprising displaying a plurality of points surrounding the joint pivot point based on a defined ligament length to identify locations on a bone where a ligament is affixable at isometric distances to the joint pivot point, using the AR headset.

34. The method as in claim 33, wherein the plurality of points is at least one of: an arc, a circle, a cylinder, a curved surface, or an irregular shape.

35. The method as in claim 31, wherein the joint pivot point is a ball joint in the person.

36. A method for referencing anatomical distance using an augmented reality (AR) headset, comprising:
determining at least one boundary of an anatomical structure for a body of a person, using a medical image data set aligned to the body of the person and an anatomical structure detection service;
registering a pointer device that has a position and orientation with respect to the anatomical structure, using the AR headset;
generating a virtual line that has an axis aligned with a lengthwise axis of the pointer device and passes through a portion of the anatomical structure; and
determining a measurement from an entry point where the virtual line enters a boundary of the anatomical structure to an additional point in the anatomical structure.

37. The method as in claim 36, further comprising displaying a numerical output for a length of the virtual line between the entry point where the virtual line enters the anatomical structure to the additional point in the anatomical structure.

38. The method as in claim 36, further comprising determining a length of a medical device to be used with the anatomical structure based on the length of the virtual line between the entry point where the virtual line enters the anatomical structure to the additional point in the anatomical structure.

39. The method as in claim 38, wherein the medical device is at least one of an implant, a screw, a needle, a stent or a trocar.

40. The method as in claim 36, wherein the additional point is a point defined by a user.

41. The method as in claim 36, wherein the additional point is where the virtual line exits from the anatomical structure.

42. The method as in claim 36, wherein the additional point is a location along the virtual line with a distance offset internal from a location where the virtual line exits the anatomical structure.

43. The method as in claim 36, further comprising determining boundaries of the anatomical structure using edge detection, feature detection, shape detection, morphometric detection or machine learning.

44. The method as in claim 36, further comprising using an optical code connected to the pointer device to enable the AR headset to register the position and orientation of the pointer device.

45. The method as in claim 36, wherein the virtual line extends from a tip of the pointer device.

46. The method as in claim 36, wherein the proximal anatomical structure is fixed in place within a 3D space.

47. A method for virtual repositioning of anatomical structure in an image data set, comprising:
aligning the image data set to a person using a marker associated with the person;
determining a joint pivot axis and angle by comparing a first pose and a second pose of movable anatomical structure of the person using the marker;
segmenting structures of anatomy in the image data set for the person to form anatomical segments in the image data set; and
rotating the anatomical segments around the joint pivot axis in the image data set, as viewed, using an AR headset.

48. The method as in claim 47, further comprising rotating the anatomical segments around the joint pivot axis to represent flexion of the movable anatomical structure in the image data set.

49. The method as in claim 47, further comprises rotating the anatomical segments around the joint pivot axis to an angle match an angle of flexion for a joint detectable by the AR headset.

50. The method as in claim 47, wherein segmenting structures of anatomy further comprises identifying bones using edge detection, feature detection, shape detection, or machine learning.

51. The method as in claim 47, further comprising registering a marker located on a movable anatomical structure of a person, wherein the marker has a first pose that includes a position and orientation in a 3D coordinate system of the AR headset.

52. The method as in claim 47, wherein the anatomical segments are moved in a 3D image space of the image data set.
